(19) United States
(12) Patent Application Publication
(10) Pub. No.: US 2015/0301012 A1
(43) Pub. Date: Oct. 22, 2015

(54) AUTOMATED LAMP WITH ULTRAVIOLET LIGHT FOR CHARACTERISING ROCKS

(71) Applicant: AXURE TECHNOLOGIES S. A., Bogota D.C. (CO)

(72) Inventors: Nestor Julio Quevedo-Cubillos, Bogota D.C. (CO); Jorge Alfonso Rozo-Herrera, Bogota D.C. (CO); Diego Francisco Jaimes-Prieto, Bogota D.C. (CO); René Guillermo Ramirez-Mora, Bogota D.C. (CO)

(21) Appl. No.: 14/650,284

(22) PCT Filed: Dec. 7, 2012
(86) PCT No.: PCT/CO2012/000004

§ 371 Date: Jun. 5, 2015

(51) Int. Cl. G01N 33/24; H04N 5/225; H04N 7/18; G01N 21/64; G01N 21/33

(52) U.S. Cl. CPC G01N 33/241; G01N 21/645; G01N 21/33; H04N 7/185; H04N 5/225; H04N 2005/2255

(57) ABSTRACT

Automated ultraviolet light lamp to characterize rocks; it consist of the following parts:
a) An electromechanical elevation system by rails (1);
b) a digital camera (2) configured inside the box (13);
c) a USB port (3) to connect the external digital camera (4) using a type C thread;
d) a laptop computer (5);
e) a control card (6), that controls the electromechanic elevation system by rails (1), daylight led ring (7), ultraviolet light (8) and DC actuator (15);
f) two Software applications to control, store all the functions, and manage the information collected by them; and
g) DC power supply (11) to energize the internal equipment.

### DESCRIPTION OF THE TECHNOLOGY FIELD

This invention refers to an Automated Ultraviolet light lamp used to characterize rocks that allows the integration—into a single equipment—of the visualization, digitalization, registry, storage, and transmission of the observations made during the drilling of oil wells to the samples used in several areas other tan geology; this allows the user to analyze easily the physical properties of the rocks either locally or remotely in order to make decisions online and in real time during the drilling of a well.

### STATE OF THE ART

During the drilling processes in search of hydrocarbons, the oil companies hire specialized service units to permanently identify, classify, register, and characterize the various properties of the rocks and underground samples extracted during the drilling, thus using various techniques and procedures related with different geology areas mentioned below.

### 1.1 Fluoroscopy in the Oil Industry

To identify the presence of crude oil, see quality using the API classification (relative density level of oil according to the American Petroleum Institute) and deduce the mobility of hydrocarbons. The continuous recording companies use the fluorescence technique developed several decades before: by stimulating substances, such as crude oil, with ultraviolet light lamps that produce electromagnetic radiation in wave lengths of 15-400 nm they can absorb that energy and radiate it again as fluorescence in longer wave lenghts that are now within the range of visible light (above 400 nm). The oil emits fluorescence in a range of colors, from matte brown for heavy grade oils and tar to a yellowish and bluish white for very light and condensed oils. This fluorescence relates to the presence of crude oil and to certain approximate API ranges depending on the color scheme emitted.

To test the samples with ultraviolet light, the company requires dark rooms/spaces in order to see clearly the luminiscent reaction of the crude oil; as a result, said ultraviolet light lamp is mostly installed in small metal or wooden boxes with a front glass viewfinder that enables users to see the fluorescence in a dark environment by blocking the external light with their head while the observe the reaction of the sample. This box is commonly referred to as Fluoroscope in the oil industry. Once users observed the reaction, they stop watching through the viewfinder; and, if they observed any luminiscent reaction, they compare the image observed and memorized visually with a color chart that correlates the approximate API of the hydrocarbon sample.

Later, the description of the observation is recorded by hand or in a worksheet or database but without the possibility of attaching quality images or photographies of the luminiscent reaction, if any. This reaction is often amplified or improved by adding ketone to the sample; this allow the sample to better react upon contact with the sample.

Some instruments include an additional white-light lamp to ease the handling of the sample inside the box without having to remove it or use the Ultraviolet Light which observation usually has well-known fully studied adverse effects on human health.

The ultraviolet light boxes or fluoroscopes have been used in the oil industry field operations for over 50 years without major changes or improvements, allowing the observation of the sample through a glass. If a sample zoom-in or close-up is required, it is necessary to use separate magnifying lenses or microscopes from the Fluoroscope, but without the possibility to stimulate the sample with Ultraviolet light. As a result, it is almost impossible to make magnified observations of the samples in field.

The transition and adaptation of the eyes when observing in conventional Fluoroscopes with UV light and then the color charts to make a correlation of API require special skills to ensure that the interpretation matches the phenomenon observed.

Although there is a big prevaling industry of ultraviolet light (bulb) lamps worldwide, the industry of Fluoroscopes with a high technology value added for the oil sector is not well developed, yet.

### 1.2 Lithological Characterization

The lithological characterization is the identification and detailed description of geological, physical, chemical, and mineralogical characteristics of a rock considering the type of rock.

Currently, for this type of descriptions it is important the direct observation of the rocks and close-up views using trinocular microscopes or other instruments that allow observing the rock in detail. The direct observation requires ensuring a white light source, preferably daylight, that does not affect the interpretation of the characteristics of the sample just as it occurs when observing using an optical instrument. When observing directly using optical aids, the description is registered in reports or records and may occasionally contain digital images that are sent afterwards to the operators' management centers or headquarters via e-mail.

Also, when chemical tests are made, it is necessary to make a direct observation at the exact moment when the expected chemical reaction occurs; this observation is subsequently registered in a report according to the operator's memory and expertise. The use of video transmissions of these tests in real time is unknown in the oil industry given the technological limitations of communications from the fields.

### 1.3 Biostratigraphy

Biostratigraphy consists of the correlation of separated stratigraphic units in time by interpreting and analyzing preserved microfossils or evidence of past life; and the number and type of fossils found allows determining a relative age of the layers.

The clear identification of the layers is helpful to evaluate interest zones for the oil exploration.

Nowadays, an expert in this technique is called to the exploration area to render biostratigraphy services. In most of the cases, the samples are selected and prepared by an assistant in order to be observed by the expert using a microscope; then, the expert reports his/her interpretion of the observations so that decisions can be taken during the process. Photos are occasionally taken from prepared plates and attached to the report; however, a correct interpretation can be validated in the field only by observing directly through the microscope.

### 1.4 Petrography.

Petrography consists of observing rocks focusing on their description, composition, and mineral structure. To observe through a microscope, rocks are cut using special equipment to obtain widths of 0.3 microns; this physical condition is required to observe through a petrographic microscope. The clear identification of minerals in a rock permits the determination of relevant properties of rocks during the oil exploration.

Currently, horizontal drilling requires petrography services more often.

An expert in this technique is then called to the field to render the services. In most of the cases, the samples are selected and prepared by an assistant in order to be observed by the expert using a petrographic microscope; then, the expert reports his/her interpretion of the observations so that a group of integrators can take decisions during the drilling process. Photos are occasionally taken from petrographic plates and attached to the report; however, a correct petrographic interpretation can be validated in the field only by observing directly through the petrographic microscope.

[This activity is not always possible] with the current communication systems and their capacity to transmit video continuously, in real time at affordable costs under the established operation budget; also, digital cameras of the microscopes in the field or the configuration of the current screens available in the field and the main offices in the city do not offer an identical image with the same definition and color to that obtained from the petrographic microscope in the field.

### 1.5 Geomechanics.

Geomechanics is the discipline that studies the mechanical characteristics of the geological materials of rocks. The studies are based on the concepts and theories of rock and ground mechanics that relate the behavior of a rock formation under the changes of effort that result from the drilling operations; although the basics of geomchanics were stated at the beginning of the [20th] century, the oil applications started to be promoted in the early 70's; therefore, this is a new discipline for the petroleum engineering.

Geomechanics uses both the field and lab experimental analyses and results together to solve particular problems.

Currently, geomechanical analyses are required more often in drilling operations. An expert is then called to the field for this purpose. In most of the cases the samples are picked from larger rock fragments than from drilled cuts; they are, in general, of 2 cms or larger and are prepared by an assistant so that they can be analyzed and classified by an expert at a later time.

The classification and analysis procedure is done visually, without the aid of instruments used to magnify the observation. The expert reports his/her interpretation of the observations so that a group of integrators can take decisions during the drilling process. Occasionally, photos are taken from the caving types and attached to the report; however, in case of particular problems such as a pega, a correct decision to solve the particular problem of the well can be validated only by observing directly the cavings.

### ADVANTAGES OF THE INVENTION

The Automated Lamp of the invention patent application presents the following advantages with respect to those of the state of the art:
It enables the local visualization of the samples visualization in two 19″ LED screens that fully protect the observers from the harmful ultraviolet rays to which they could be exposed when using conventional lamps or Fluoroscopes without UV filters.
Since the equipment has two folding True Color screens, it allows a reliable local visualization by two people at the same time to interact upon the observations, as opposed to the conventional fluoroscopes in which a single user can observe the samples.
The use of LED screens to visualize the fluorescence helps the users to avoid limiting their visual field to a totally dark space, thus eliminating the adaptation process of the pupils when users withdraw from the conventional fluroscope viewfinders to compare the image observed with the color correlation charts.
Since no adaptation of the observers' eyes to different light conditions (darkness-clarity) is required, the time the brain has to keep the image or tone observed in the memory decreases substantially, thus enabling a much more immediate correlation between the observation and the pattern to be compared with (color chart).
By having visualization screens at head level and the possibility to rotate them on the vertical axis, it improves the lumbar and cervical postures of the observer when making continuous observations in
   comparison with the curved position of the observer's back when using conventional fluoroscopes.
It allows the observer to move the sample (by bringing it closer or moving it away) using electromechanics without the need to remove the simple from the equipment, as opposed to the conventional fluoroscopes in which the distance between the sample and the viewfinder is fixed and users must introduce their hands to bring the sample closer or move it away while the UV light is on; this implies that the skin will be dangerously exposed to the light.
It has an automatic switch-off system of the UV light that activates when any of the covers are opened to Access to the samples; the conventional fluoroscopes do not have this type of protection.
It has a white led light ring that improves the distribution of light onto the sample thus eliminating the possibility to produce the shadows that occur in conventional fluoroscopes with daylight lamps placed in any of the ends of the equipment.
The configuration of the daylight in the ring allows a proper control of the intensity of light and the shadows to highlight any physical characteristics of the rock. Conventional fluoroscopes cannot control the distribution of light.
Unlike conventional lamps which cannot control the strobe light, the Automated Ultraviolet Light Lamp can also control the frequency with which the daylight ring is switched on.
It provides a digital photographic record of the observations at the moment of the samples' fluorescence or when they react to different chemical reagents. Conventional fluoroscopes do not allow a digital record of said phenomena.
It allows a real-time transmission of what is being observed in the equipment with hi-def images that can be interpreted in remote places by one or more observers located in different parts of the world, unlike the conventional lamps with do not integrate a real-time transmission system.
It admits real-time transmissions, captures, recording, storage, and local visualization of the true color and high definition images that are being observed in equipment near to the lamp (5 meters or less) like petrographic microscopes, trinocular and biological microscopes, and samples that are being observed directly without an equipment and related to the petrography, lithology, biostratigraphy and/or geomechanical processes, respectively. No Ultraviolet Light lamp is designed with this functionality. Modern microscopes have cameras that are not usually compatible with other optical devices and never with Ultraviolet Light Fluoroscopes such as those used in Mud Logging services.
It enables the digital storage of video images generated during the analyses of fluorescence, Ilthology, petrography, biostratigraphy or geomechanics and indexed per date of capture in a massive digital storage medium.
It allows the digital registration of the identification and description of the properties of the rocks or the oil found by using a software application designed to operate once the samples are inside the lamp or any of the peripheral optical instruments of the automatic lamp.
The remote real-time transmission of images enables experts in different techniques (petrography, geomechanics, biostratigraphy, geology, palynology, or geochemistry) to make accurate interpretations as if they were in the field; this reduces the time spent in transportation and saves costs associated to critical decision making.
The remote real-time transmission of images can be performed in reverse by installing the automatic lamp at the interpreters' office, in the city, so that when the experts are in the field, they can do the correlation and interpretation of samples preserved at their mineral collection.
Digital storage of the images captured accepts reviewing processed samples at any moment to be compared in new projects or detect mistakes made in yet completed operations.

It enables the construction of an image bank that contains all the rock samples analyzed during the drilling process. This file is a backup system that is used to make partial analyses when physical samples are lost.

### LIST OF ATTACHED FIGURES

FIG. 1 Front perspective view of the Automated Ultraviolet Light Lamp of the present invention patent application

FIG. 2 Front view of the Automate Ultraviolet Light Lamp.

FIG. 3 Side sectional view of the Automated Ultraviolet Light Lamp.

FIG. 4 Front sectional view of the Automated Ultraviolet Light Lamp.

FIG. 5 Rear perspective view of the Automated Ultraviolet Light Lamp.

FIG. 6 Top perspective view of the interior of the Automated Ultraviolet Light Lamp.

FIG. 7 Bottom perspective view of the interior of the Automated Ultraviolet Light Lamp.

FIG. 8 Block diagram with the interconnection between the various functional parts of the Automated Ultraviolet Light Lamp described in the present invention patent application.

### DESCRIPTION OF THE INVENTION

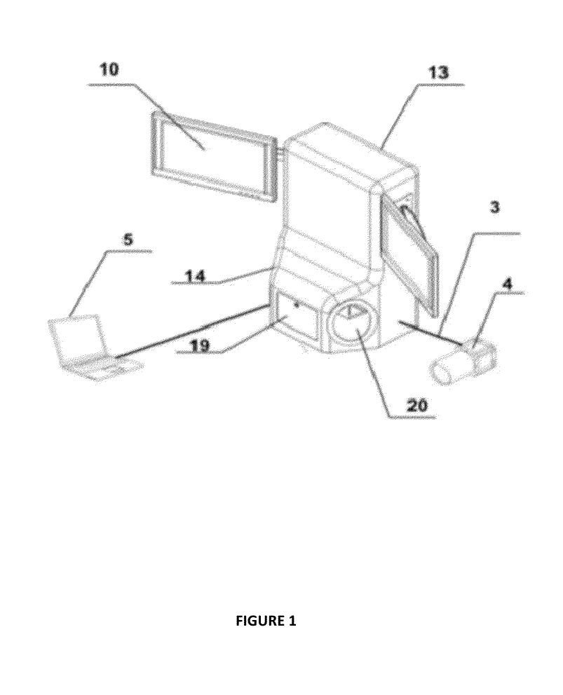

The Automated Ultraviolet Light Lamp for the characterization of rocks that is described in the present invention patent application, as it is shown in FIG. 1 above is a box (13) or a body in the shape of a stretched regular vertical cuboid with two screens (10) at the level of an average height user; at the bottom it has a chassis (14) in the shape of an irregular cuboid that has three access points: one to introduce samples, a front hatch/gate (19), and two openings (20) at the chassis side wall (14) that the observers use to introduce their arms; it is specially designed/crafted to ease the handling of samples and the execution of the activities related to the several routines of the various geology fields. As a safety measure, when the user opens the front hatch (19) to introduce or withdraw sample, the Ultraviolet lights switch off.

The Automated Ultraviolet Light Lamp consists of the following parts shown in the attached figures:
a) An electromechanical elevation system by rails (1) that hold a base (17) containing a daylight led ring in the center of the bottom part (7), an ultraviolet light lamp (8) placed on the edges of the base (17); this system is specially built in to focus closely on the samples inside the equipment;
b) a digital camera (2) configured inside the box (13) to capture images inside the equipment;
c) a USB port (3) to connect an external digital camera (4), (FIG. 1) with a type C thread specially adapted to capture images using other optical equipment (peripheral equipment);
d) a laptop computer (5) with an input/output Interface for the different peripherals; this laptop executes the Automated Ultraviolet Light Lamp software application;
e) a control card (6), that controls the electromechanic elevation system by rails (1), the daylight led ring (7), the ultraviolet light (8) and a DC actuator (15);
f) two software applications specialized in controlling, storing functions, and managing the information that they capture;
g) A DC power supply unit (11) to energize the internal equipment.

### DETAILED DESCRIPTION OF THE INVENTION

### Electromechanical Elevation System by Rails (1)

It allows the users to bring or move the internal camera (2) closer to or away from the sample using command lines programmed from the software; it mainly consists of a DC actuator (15), and a side rail system (1).

### Control Card (6)

It is designed by the applicant to interact with the software in the laptop (5) and allows the user to operate the daylight led ring (7), ultraviolet light (8), and DC actuator (15). The control card (6) consists mainly of a micro controller (12), a USB integrated circuit (18) to create a virtual serial connection with the calculation equipment calculation, and other integrated circuits to control the engines and power stages.

### Daylight LED Ring (7)

It is an electronic card (7) designed by the applicant; it contains 104 5 mm leds distributed in a round manner, in groups of 13 leds. Their intensity, on/off frequency, illuminated areas, and location from the control software application is controlled.

### Sample Management Software.

It is a software developed by the applicant in web design languages; it allows the administration and storage of the images captured by the Automated Ultraviolet Light Lamp, the registration of information, and the generation of reports under a Web platform.

### Control Software.

It is made by the applicant to allow the observers or users to control the ultraviolet light (8) and the daylight ring (7) so that they can illuminate segments in a different sequence order; obtain several observation perspectives and highlight details that could not be obtained using a conventional lamp; control the illumination frequency of the daylight led ring (7) between 1 Hz and 60 Hz; control the intensity of the light to obtain the best option to appreciate the different parts or compounds of the rock samples and others; control the vertical movement of the internal digital camera (2) to allow real close-ups of the sample without having to use the zoom and preserving the quality of the image; this allows

the user to position the camera along a specific distance range.

Each attached Figure show the following:

FIG. 1 is a front perspective view of the Automated Ultraviolet Light Lamp described in the present invention patent application that shows: the box (13), the chassis (14) with the front hatch (19), two openings (20), and two screens (10).

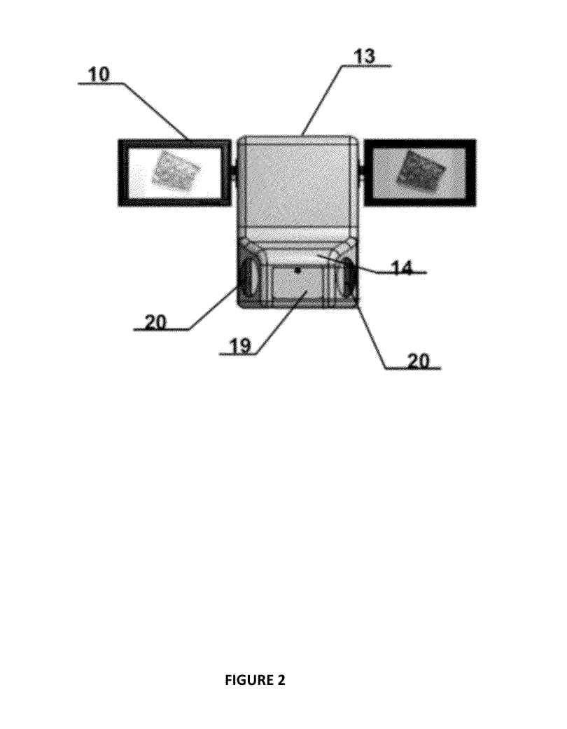

FIG. 2 is the front view of the Automated Ultraviolet Light Lamp of the invention and shows: the box (13), the chassis (14) with the front hatch (19), two openings (20), and two screens (10).

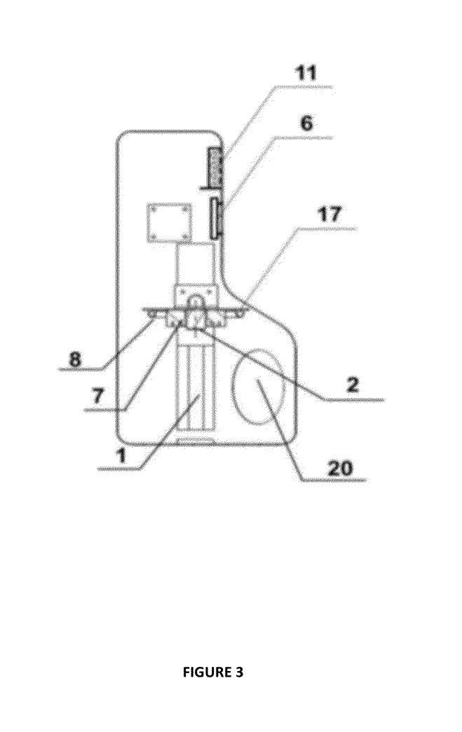

FIG. 3 is the side sectional view of the Automated Ultraviolet Light Lamp of the invention and shows: the electromechanic elevation system by rails (1), digital camera (2), base (17), daylight led ring (7), ultraviolet light lamp (8), opening (20), control card (6), and DC power supply unit (11).

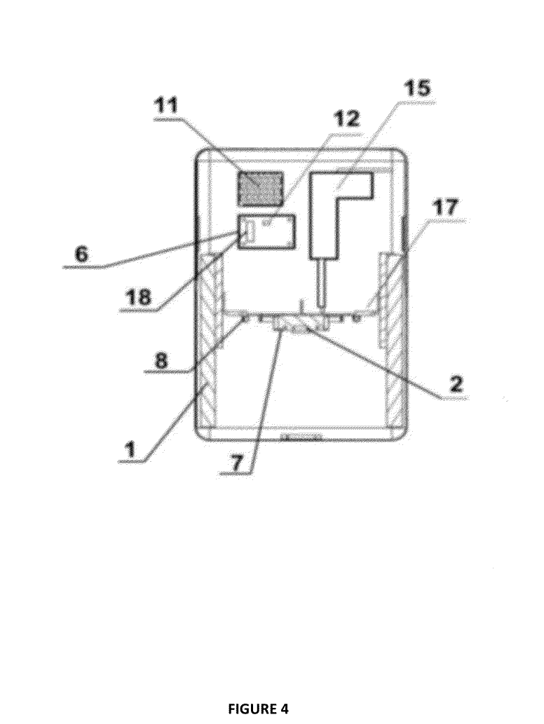

FIG. 4 is the front sectional view of the Automated Ultraviolet Light Lamp of the invention that shows:

the electromechanical elevation system by rails (1), digital camera (2), base (17), daylight led ring (7), ultraviolet light (8), control card (6), DC power supply unit (11), microcontroller (12) inside the control card (6), DC actuator (15), and USB integrated circuit (18).

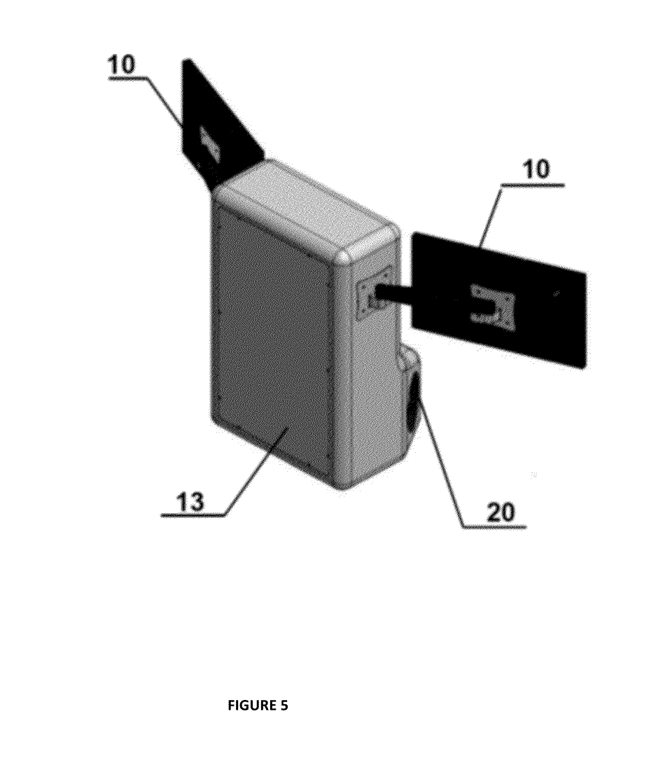

FIG. 5 corresponds to the rear perspective view of the Automated Ultraviolet Light Lamp of the invention showing: two screens (10), the box (13), and the opening (20).

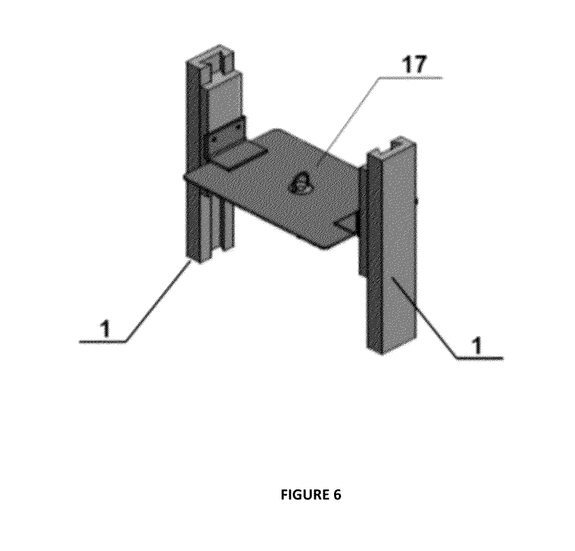

FIG. 6 shows the top perspective view of the interior of the Automated Ultraviolet Light Lamp of the invention showing: the electromechanical elevation system by rails (1) and the base (17).

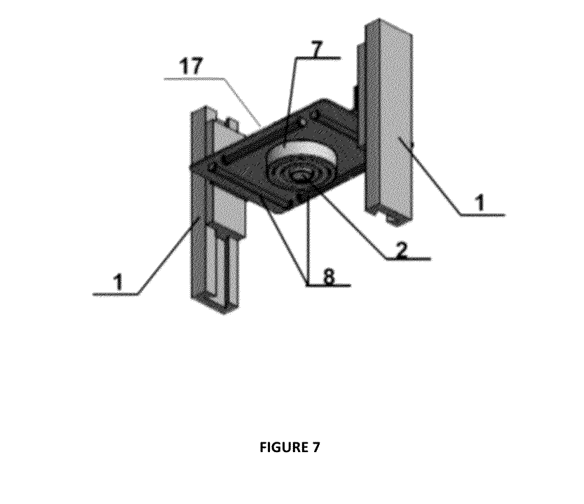

FIG. 7 is the bottom perspective view of the interior of the Automated Ultraviolet Light Lamp of the invention and shows: the electromechanic elevation system by rails (1), digital camera (2), base (17), daylight led ring (7), and ultraviolet light lamp (8).

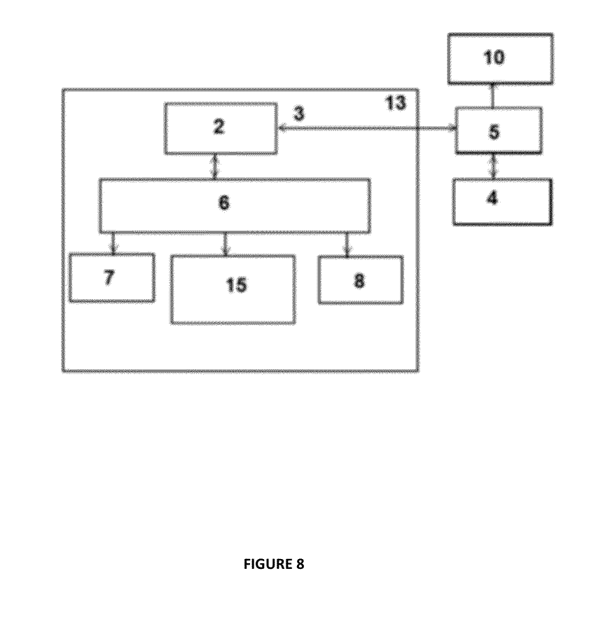

FIG. 8 corresponds to the interconnection using a general block diagram f the functional parts of the Automated Ultraviolet Light Lamp described in the present invention patent application and showing: the box (13) that contains the daylight led ring (7), DC actuator (15), and ultraviolet light lamps (8) that depend directly on the control card (6). The digital camera (2) is connected to the control card (6), in a two-way configuration; the camera is interconnected to the laptop computer (5) outside of the box (13) using a USB port (3). Two led screens (10) are connected to the laptop computer (5) and an external digital camera (4) is connected in a bidirectional configuration.

1. Automated ultraviolet light lamp for the characterization of rocks CHARACTERIZED BECAUSE the lamp in the upper part is a box (13) or body in the shape of a regular stretched an vertical cuboid with two screens (10) and a chassis (14) in the bottom part in the shape of an irregular cuboid that can be accessed in three different points to introduce samples, a front hatch (19), and two openings (20) in the side wall to allow the obserers introduce their arms; the lamp consists of the following parts:
a) An electromechanic elevation system by rails (1) that supports a base (17) which holds a daylight led ring (7) in the center of the bottom part, an ultraviolet light lamp (8) on the edges of a base (17);
b) a digital camera (2) configured inside the box (13);
c) a USB port (3) to connect the external digital camera (4) with a type C thread and capture images of other optical equipment;
d) a laptop computer (5) with the input/output interface for the different peripherals; the laptop executes the software application of the equipment.
e) a control card (6), that controls the electromechanic elevation system by rails (1), daylight led ring (7), ultraviolet light (8) and DC actuator (15);
f) two Software applications to control, store all the functions, and manage the information collected by them;
g) a DC power supply unit (11) to energize the internal equipment.

2. Automated Ultraviolet Light Lamp of claim 1, CHARACTERIZED BECAUSE the internal camera (2) can be moved closer to or away from the sample using the electromechanic system that uses commands programmed from the equipment software; also, it contains a DC actuator (15) and a side rail system (1).

3. Automated Ultraviolet Light Lamp of claim 1, CHARACTERIZED BECAUSE the control card (6) consists mainly of a micro controller (12) and an integrated USB circuit (18), and operates the daylight led ring (7), ultraviolet light (8), and DC actuator (15).

4. Automated Ultraviolet Light Lamp of claim 1 CHARACTERIZED BECAUSE the lamp box (13) contains a daylight led ring (7), DC actuator (15), ultraviolet light lamps (8) that depend directly on the control card (6); the digital camera (2) is connected to the control card (6), in a two-way configuration; the camera is interconnected to the laptop computer (5) outside of the box (13) using a USB port (3). Two led screens (10) are connected to the laptop computer (5), and an external digital camera (4) is connected in a bidirectional configuration.
